(19) United States
(12) Patent Application Publication
(10) Pub. No.: US 2006/0154936 A1
(43) Pub. Date: Jul. 13, 2006

(54) Use of n-'5-'4-(4-methylpiperaziomethyl)-benzoylamido!-2-methylphenyl!-4-(3-pyridyl)2-pyridine-amine for the treatment of pulmonary hypertension

(76) Inventor: Joseph Alexander Lasky, New Orleans, LA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/532,297

(22) PCT Filed: Oct. 20, 2003
(86) PCT No.: PCT/IB03/04671

§ 371 Date: Aug. 24, 2005

Related U.S. Application Data
(60) Provisional application No. 60/421,318, filed on Oct. 25, 2002.

(51) Int. Cl. A61K 31/506

(52) U.S. Cl. 514/252.18

(57) ABSTRACT

The present invention pertains to the use of 4-(4-methylpiperazin-1-ylmethyl)-N-[4-methyl-3-(4-pyridin-3-yl)pyrimidin-2-ylamino)phenyl]-benzamide of the formula I 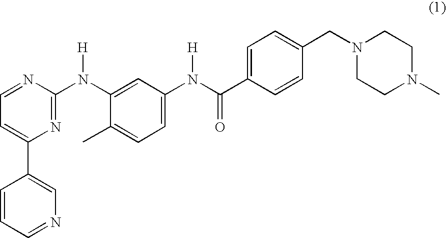 or a pharmaceutically acceptable salt thereof for the manufacture of a medicament for the treatment of pulmonary hypertension.

The invention relates to the use of 4-(4-methylpiperazin-1-ylmethyl)-N-[4-methyl-3-(4-pyridin-3-yl)pyrimidin-2-ylamino)phenyl]-benzamide (hereinafter: “COMPOUND I”) or a pharmaceutically acceptable salt thereof for the manufacture of pharmaceutical compositions for use in the treatment of pulmonary hypertension, to the use of COMPOUND I or a pharmaceutically acceptable salt thereof in the treatment of pulmonary hypertension, and to a method of treating warm-blooded animals including humans suffering from pulmonary hypertension, by administering to a said animal in need of such treatment an effective dose of COMPOUND I or a pharmaceutically acceptable salt thereof.

Pulmonary hypertension, generally defined as a pulmonary artery systolic pressure exceeding 25 mmHg, is either idiopathic in nature (primary pulmonary hypertension) or a manifestation of many different disorders (secondary pulmonary hypertension) and may have many varying etiologies. However, regardless of the initiating factors, the disease pathways and characteristics are similar. Patients with pulmonary hypertension generally present with dyspnea, precordial discomfort, and angina. On physical examination, cyanosis, oedema, jugular venous distension as well as right ventricular heave, right sided S3 gallop and loud SP2 are often present.

Pulmonary hypertension is often a progressive disease in which there is a gradual increase in vascular resistance that leads to right heart failure and may be fatal. Histological examination of tissue samples from patients with pulmonary hypertension shows intimal thickening, as well as smooth muscle cell hypertrophy, especially for those vessels <100 μm diameter. Endothelial cells play a central role in the disease process. This is not surprising since many of the humoral factors known to both positively and negatively affect the disease either are synthesized by these cells or act upon them. Damage of endothelial cells often initiates the disease. The humoral factors that potentiate pulmonary hypertension are generally vasoconstrictors, such as endothelin-1 (ET-1), which increases pulmonary resistance in part by reducing vessel caliber, whereas factors antagonizing the disease are generally vasodilators, such as nitric oxide (NO), which reduce arterial pressure.

Primary pulmonary hypertension (PPH) is a rare disease entity with unknown etiology. The clinical course is generally one of relentless progression toward death. Connective tissue diseases are occasionally complicated by secondary pulmonary hypertension (PH). Like PPH, secondary PH can also significantly affect quality of life and hasten death in patients with connective tissue diseases. A number of vasodilating agents, including adenosine, nitroprusside, prostaglandin I₂, calcium channel blockers, and inhaled nitric oxide, have been tested during cardiac catheterization for their acute hemodynamic effects and to form the basis for long-term therapy consideration. However, the efficacy of pulmonary vasodilator therapy has been limited because of the lack of potent pulmonary vasodilating agents that selectively vasodilate for the pulmonary vasculature and because of fixed pulmonary vascular “remodelling”.

Primary pulmonary hypertension and pulmonary hypertension associated with collagen vascular diseases such as scleroderma are notoriously difficult to treat. Long-term anticoagulation, calcium channel blockers, intravenous infusion, and inhalation of prostacyclin or its derivatives are all of benefit but of quite limited efficacy.

Primary pulmonary hypertension is an uncommon disease characterized by increased pulmonary artery pressure and pulmonary vascular resistance. The incidence has been estimated at approximately 2 cases per million. There is a female-to-male preponderance (1.7:1), with patients most commonly presenting in the third and fourth decades, although the age range is from infancy to greater than 60 years. Because the predominant symptom of primary pulmonary hypertension is dyspnea, which can have an insidious onset in an otherwise healthy person, the disease is typically diagnosed late in its course. By that time, the clinical and laboratory findings of severe pulmonary hypertension are usually present. The histopathology of primary pulmonary hypertension is not pathognomonic for the disease but represents a pulmonary arteriopathy that is observed in pulmonary hypertension from a variety of causes. A wide spectrum of vascular abnormalities involving the endothelium, smooth muscle cells, and extracellular matrix is present. Heterogeneity with respect to these abnormalities is often seen from patient to patient, and within patients. The most common features noted are, e.g. medial hypertrophy and plexiform lesions. In most patients, varying degrees of these abnormalities can be found.

Rare variant forms of primary pulmonary hypertension also exist.

Pulmonary venoocclusive disease is a rare and distinct pathologic entity, found in fewer than 10% of patients with primary pulmonary hypertension. These patients may develop orthopnea that can mimic left ventricular failure.

Pulmonary capillary hemangiomatosis is also a very rare form of primary pulmonary hypertension. These patients often have hemoptysis as a clinical feature.

Secondary pulmonary hypertension may reflect “remodelling” of the arterial wall with abnormalities of elastic fibers, and medial hypertrophy that result in vascular stiffness and reduced vasodilator responsiveness. Although possibly reversible over time, i.e. months, the pulmonary hypertension attributable to structural remodelling is generally referred to as “fixed” because it is not rapidly responsive, i.e. minutes to days, to reversal with pharmacological maneuvers.

The instant invention is a response to the need for an alternative therapy in the treatment of pulmonary hypertension, especially primary pulmonary hypertension and secondary pulmonary hypertension.

It has now surprisingly been demonstrated that pulmonary hypertension can be successfully treated with COMPOUND I, or pharmaceutically acceptable salt thereof.

The present invention concerns the use of 4-(4-methylpiperazin-1-ylmethyl)-N-[4-methyl-3-(4-pyridin-3-yl)pyrimidin-2-ylamino)phenyl]-benzamide having the formula I 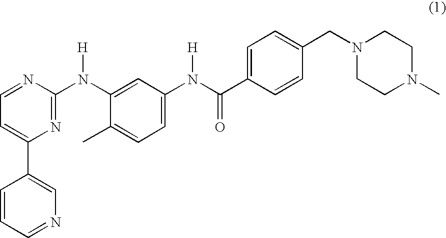
or a pharmaceutically acceptable salt thereof, for the manufacture of a medicament for treating pulmonary hypertension.

4-(4-methylpiperazin-1-ylmethyl)-N-[4-methyl-3-(4-pyridin-3 -yl)pyrimidin-2-ylamino)phenyl]-benzamide or a pharmaceutically acceptable salt or β-crystal form thereof will be referred herein as COMPOUND I (also known as “Imatinib” [International Non-proprietary Name]).

The preparation of COMPOUND I and the use thereof, especially as an anti-tumor agent, are described in Example 21 of European patent application EP-A-0 564 409 hereby incorporated by reference, which was published on 6 Oct. 1993, and in equivalent applications and patents in numerous other countries, e.g. in U.S. Pat. No. 5,521,184 and in Japanese patent 2706682.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The monomethanesulfonic acid addition salt of COMPOUND I (hereinafter “COMPOUND I mesylate” or “imatinib mesylate” or “COMPOUND I monomethanesulfonate”) and a preferred crystal form thereof, e.g. the β-crystal form, are described in PCT patent application WO99/03854 published on Jan. 28, 1999.

Possible pharmaceutical preparations, containing an effective amount of COMPOUND I or a pharmaceutically acceptable salt thereof are also described in WO99/03854 hereby incorporated by reference.

The present invention particularly concerns the use of COMPOUND I or a pharmaceutically acceptable salt thereof for the manufacture of a medicament for treating pulmonary hypertension, especially primary pulmonary hypertension and secondary pulmonary hypertension. The present invention pertains to the use of COMPOUND I or a pharmaceutically acceptable salt thereof for the preparation of a medicament for the treatment of pulmonary hypertension not caused or not accompanied by pulmonary fibrosis.

The invention pertains to a pharmaceutical preparation for the treatment of pulmonary hypertension, especially primary pulmonary hypertension and secondary pulmonary hypertension, comprising 4-(4-methylpiperazin-1-ylmethyl)-N-[4-methyl-3-(4-pyridin-3-yl)pyrimidin-2-ylamino)phenyl]-benzamide having the formula I.

The term treatment” as used herein means curative treatment and prophylactic treatment.

The term “curative” as used herein means efficacy in treating ongoing episodes of pulmonary hypertension.

The term “prophylactic” means the prevention of the onset or recurrence of pulmonary hypertension.

Depending on species, age, individual condition, mode of administration, and the clinical picture in question, effective doses, for example daily doses of about 100-1000 mg, preferably 200-600 mg, especially 400 mg of COMPOUND 1, are administered to warm-blooded animals of about 70 kg bodyweight. For adult patients with unrespectable pulmonary hypertension, a starting dose corresponding to 400 mg of COMPOUND I free base daily can be recommended. For patients with an inadequate response after an assessment of response to therapy with a dose corresponding to 400 mg of COMPOUND I free base daily, dose escalation can be safely considered and patients may be treated as long as they benefit from treatment and in the absence of limiting toxicities.

The invention relates also to a method for administering to a human subject having pulmonary hypertension, a pharmaceutically effective amount of COMPOUND I or a pharmaceutically acceptable salt thereof to the human subject. Preferably, COMPOUND I or a pharmaceutically acceptable salt thereof is administered once daily for a period exceeding 3 months. The invention relates especially to such method wherein a daily dose of COMPOUND I mesylate corresponding to 100 to 1000 mg, e.g. 200 to 800 mg, especially 400-600 mg, preferably 400 mg, of COMPOUND I free base is administered.

According to the present invention, COMPOUND I is preferably in the form of an acid addition salt, e.g. monomethanesulfonate salt, e.g. in the β-crystal form of the monomethanesulfonate salt.

The invention relates to a method of treating a warm-blooded animal, especially a human, suffering from pulmonary hypertension, comprising administering to the animal a combination which comprises (a) N-[5-[4-(4-methyl-piperazino-methyl)-benzoylamido]-2-methylphenyl]-4-(3-pyridyl)-2-pyrimidine-amine (designated hereinafter as COMPOUND I) and (b) at least one compound selected from compounds indicated for the treatment of pulmonary hypertension such as calcium channel antagonists, e.g. nifedipine, e.g. 120 to 240 mg/d, or diltiazem, e.g. 540 to 900 mg/d, prostacyclin, adenosine, inhaled nitric oxide, anticoagulants, e.g. warfarin, digoxin, endothelin receptor blockers, e.g. bosentan, phosphodiesterease inhibitors, e.g. Viagra, norepinephrine, angiotensin-converting enzyme inhibitors e.g. enalapril or diuretics; a combination comprising (a) and (b) as defined above and optionally at least one pharmaceutically acceptable carrier for simultaneous, separate or sequential use, in particular for the treatment of pulmonary hypertension; a pharmaceutical composition comprising such a combination; the use of such a combination for the preparation of a medicament for the delay of progression or treatment of pulmonary hypertension, e.g. primary pulmonary hypertension and secondary pulmonary hypertension; and to a commercial package or product comprising such a combination.

It can be shown by established test models that the COMPOUND I or a pharmaceutically acceptable salt thereof, results in a more effective prevention or preferably treatment of pulmonary hypertension. COMPOUND I or a pharmaceutically acceptable salt thereof has significant fewer side effects as a current therapy. Furthermore, COMPOUND I or a pharmaceutically acceptable salt thereof, results in beneficial effects in different aspect of pulmonary hypertension such as, e.g. medial hypertrophy.

COMPOUND I, or a pharmaceutically acceptable salt thereof, shows an unexpected high potency to prevent or eliminate pulmonary hypertension because of its unexpected multifunctional activity, and its activity on different aspects of pulmonary hypertension.

The person skilled in the pertinent art is fully enabled to select a relevant test model to prove the hereinbefore and hereinafter indicated therapeutic indications and beneficial effects (i.e. good therapeutic margin, and other advantages mentioned herein). The pharmacological activity is, for example, demonstrated by in vitro and in vivo test procedures, or in a clinical study as essentially described hereinafter. The following Example illustrates the invention described above, but is not, however, intended to limit the scope of the invention in any way.

### EXAMPLE 1

This study is designed to investigate the effects of PDGF receptor signal transduction blockade on the development of hypoxia-induced pulmonary hypertension using a PDGF receptor-selective tyrosine kinase inhibitor, e.g. COMPOUND I, COMPOUND I mesylate.

The methodology as described by Ortiz L A, Champion H C, Lasky J A and col. (Am. J. Physiol. Lung Cell. Mol. Physiol. June 2002; 282(6):L1209-21) can be implemented to prove the herein described therapeutic use and beneficial effects.

Pulmonary arterial pressure (PAP) can be measured in anesthetized mice with the use of a single-lumen catheter (Nu-Med, Hopkinton, N.Y.). The catheter (145 mm in length, 0.25 mm OD) has a specially curved tip to facilitate passage through the right heart, main pulmonary artery, and the left or right pulmonary artery. Immediately after placement of the pulmonary catheter (30 min in average), pressure in the main pulmonary artery is measured with a pressure transducer (Schneider/Namic, Glenns Falls, N.Y.), and mean PAP is derived electronically and recorded continuously. For the determination of pulmonary arterial wedge pressure, the catheter is advanced to the left or right pulmonary artery and wedged with continuous measurement of the pressure waveform.

Lung morphology and evaluation of right ventricular hypertrophy can be performed as below. The heart is perfused with 0.9% NaCl to remove residual blood, and the right lung is fixed in situ for 2 h by intratracheal instillation of 10% neutral formalin (Sigma, St. Louis, Mo.) at a constant pressure of 30 cmH₂O and was preserved in fixative for 24 h. Lung tissues are then sectioned sagittally and embedded in paraffin. Sections (4 μm thick) are generated and mounted on positively charged slides (Fisher Scientific, Pittsburgh, Pa.). Slides are stained with hematoxylin-eosin for light microscopic examination. Immediately after death, hearts are resected to evaluate right ventricular hypertrophy. The atria are removed up to the plane of the atrial-ventricular valves. The right ventricle (RV) free wall is then dissected free of the left ventricle (LV) and septum. The RV and LV plus septum are weighed and the RV-to-LV+septum ratio is calculated.

Ten Spraque-Dawley rats are exposed to hypobaric-hypoxic (HH) conditions and ten ambient air (AA) animals are used as controls. The rats in both conditions are randomized to receive intraperitoneal injections with either once daily COMPOUND I (50 mg/kg) or placebo for 3 weeks. Right cardiac catheterizations are used to evaluate the mean pulmonary arterial pressure (mPAP). Subsequently, the rats are sacrificed for the evaluation of their medial hypertrophy index (% wall thickness of the pulmonary artery), and right ventricular hypertrophy (right ventricle/left ventricular +septum weight ratio; RV/LV+S).

Results:

| | HH/CPDI | HH/Placebo | AA/CPD I | AA/Placebo |
|---|---|---|---|---|
| mPAP (mmHg) | 19.50 ± 1.32 | 31.75 ± 1.79 | 16.20 ± 1.93 | 15.40 ± 0.51 |
| % wall | 14.77 ± 0.73 | 21.20 ± 1.24 | 10.32 ± 0.33 | 9.57 ± 0.41 |
| thickness | | | | |
| RV/LV + S(%) | 35.85 ± 1.29 | 47.44 ± 3.08 | 24.60 ± 1.08 | 26.58 ± 1.67 |

Conclusions: Treatment with COMPOUND I (CPD I) reduced hypoxic-induced pulmonary hypertension in rats by 80% (p=0.002), RV hypertrophy by 46% (p=0.006), and lowered the index of % wall thickness by 59% (p=0.001).

Taken together, these results suggest that COMPOUND I, e.g. COMPOUND I mesylate, has an unexpected potential for the treatment of pulmonary hypertension.

### EXAMPLE 2

### Capsules with 4-[(4-methyl-1-piperazin-1-ylmethyl)-N-[4-methyl-3-[[4-(3-pyridinyl)-2-pyrimidinyl]amino]phenyl]benzamide monomethanesulfonate, or its β-crystal Form

Capsules containing 119.5 mg of the compound named in the title (=COMPOUND I mesylate) corresponding to 100 mg of COMPOUND I (free base) as active substance are prepared in the following composition:

| | | |
|---|---|---|
| COMPOUND I mesylate | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

### EXAMPLE 3

### Capsules with 4-{(4-methyl-1-piperazin-1-ylmethyl)-N-[4-methyl-3-[[4-(3-pyridinyl)-2-pyrimidinyl]amino]phenyl]benzamide monomethanesulfonate, b-crystal Form

Capsules containing 119.5 mg of SALT I corresponding to 100 mg of COMPOUND I (free base) as active substance are prepared in the following composition:

| | | |
|---|---|---|
| Active substance | 119.5 | mg |
| Avicel | 200 | mg |
| PVPPXL | 15 | mg |
| Aerosil | 2 | mg |
| Magnesium stearate | 1.5 | mg |
| | 338.0 | mg |

The capsules are prepared by mixing the components and filling the mixture into hard gelatin capsules, size 1.

These examples illustrate the invention without in any way limiting its scope.

1. (canceled)

2. The method according to claim 10, wherein the pulmonary hypertension is selected from primary pulmonary hypertension or secondary pulmonary hypertension.

3. The method according to claim 10 wherein a daily dose of 100 to 1000 mg of COMPOUND I of the formula I is administered to an adult human.

4. The method according to claim 3 wherein the daily dose is administered for a period exceeding three months.

5. The method according to claim 10 wherein the pulmonary hypertension is not caused or accompanied by pulmonary fibrosis.

6. The method according to claim 10 wherein N-{5-[4-(4-methyl-piperazino-methyl)-benzoylamido]-2-methylphenyl)-4-(3-pyridyl)-2-pyrimidine-amine of the formula I is in the form of a pharmaceutically acceptable acid addition salt.

7. The method according to claim 6 wherein N-{5-[4-(4-methyl-piperazino-methyl)-benzoylamido]-2-methylphenyl)-4-( 3 -pyridyl )-2-pyrimidine-amine of the formula I is in the form of the monomethanesulfonate salt.

8. A combination which comprises (a) N-{5-[4-(4-methyl-piperazino-methyl)-benzoylamido]-2-methylphenyl}-4-(3-pyridyl)-2-pyrimidine-amine (designated hereinafter as COMPOUND I) and (b) at least one compound selected from the group comprising calcium channel antagonists, anticoagulants, endothelin receptor blockers, angiotensin-converting enzyme inhibitors, diuretics, and phosphodiesterease inhibitors for the manufacture of a medicament for the treatment of pulmonary hypertension.

9. The combination according to claim 8 wherein (b) is at least one compound selected from the group comprising nifedipine, diltiazem, enalapril prostacyclin, adenosine, inhaled nitric oxide, warfarin, digoxin, bosentan, Viagra, and norepinephrine.

10. A method of treating humans suffering from pulmonary hypertension which comprises administering to a said human in need of such treatment a dose, effective against pulmonary hypertension, of N-15-[4-(4-methyl-piperazino-methyl)-benzoylamido]-2-methylphenyl)-4-(3-pyridyl)-2-pyrimidine-amine of the formula I 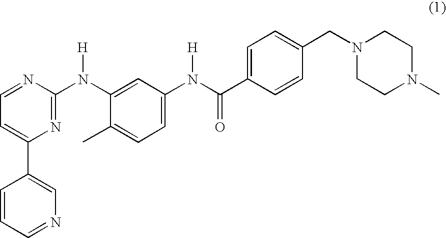
or a pharmaceutically acceptable salt thereof.
